(19) United States
(12) Patent Application Publication
(10) Pub. No.: US 2022/0241199 A1
(43) Pub. Date: Aug. 4, 2022

(54) CANNABINOID EMULSION COMPOSITION AND METHOD OF MANUFACTURE

(71) Applicant: Douglas Gore, Carlsbad, CA (US)

(72) Inventor: Douglas Gore, Carlsbad, CA (US)

(21) Appl. No.: 17/164,658

(22) Filed: Feb. 1, 2021

(51) Int. Cl. A61K 9/107; A61K 31/05; A61K 31/352; A61K 47/14; A61K 47/44; A61K 47/22; A61K 47/10; A61K 47/12

(52) U.S. Cl. CPC A61K 9/1075; A61K 31/05; A61K 31/352; A61K 47/14; B82Y 5/00; A61K 47/22; A61K 47/10; A61K 47/12; A61K 47/44

(57) ABSTRACT

A method for forming an emulsion containing a cannabinoid includes the steps of first providing a composition comprising water, vitamin E TPGS, a cannabinoid, and a carrier oil, in a high pressure container. The composition is initially opaque. The composition is then heated to a temperature between 50-120 degrees Celsius, the heating increasing the pressure in the high pressure container to greater than 1 ATM. The high pressure container is then cooled, while mixing, until the composition turns clear, and a micelle water emulsion is formed with particles under 100 nm in particle size.

### BACKGROUND OF THE INVENTION

### Field of the Invention

This invention relates generally to cannabinoid compositions, and more particularly to a water soluble cannabinoid emulsion composition and method of manufacture.

### Description of Related Art

The word “Cannabis” refers to a genus of flowering plants. Plants of genus Cannabis include several species. including Cannabis sativa, Cannabis indica, and Cannabis ruderalis. Plants from genus Cannabis are useful a wide variety of medicinal purposes, as well as recreational activities.

According to some accounts. Cannabis is composed of at least 483 known chemical compounds, which include cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, and non-cannabinoid phenols.

Cannabinoids are of particular interest for research and commercialization. Most extractions of Cannabis plant matter aim to extract cannabinoids, particularly tetrahydrocannabinol (THC). THC is useful for relieving pain, treating glaucoma, relieving nausea, and a wide variety of other uses. Usually, cannabinoids are extracted from the Cannabis plant as part, of a crude mixture, combined with other chemical compounds found in the Cannabis plant.

Current methods of administration cannabinoids fail to take full advantage of cannabinoid properties. For example, burning plant matter and inhaling the vapor does not allow for selection of certain cannabinoids for their certain desired benefit. One can choose a plant with certain known properties, e.g., THC content, but there is still little to no control over is selecting individual cannabinoids. Inhaling smoke also leads to many harmful and toxic compounds introduced into the body.

There exists a need for new cannabinoid formulations. In particular, there exists a need for water soluble cannabinoid formulations. Additionally, there exists a need for methods for producing aqueous cannabinoid formulations that are superior to the prior art. Furthermore, there exists a need for making formulations with increased permeability into the bloodstream. Also, there also exists a need for Cannabis formulations which provide increased bioavailability of cannabinoids. These aqueous cannabinoid formulations enhance the solubility and absorption of lipophilic ingredients. There is a need to increase and improve oral bioavailability and intracellular uptake of lipophilic ingredients. There is a need to increase and improve topical bioavailability and skin absorption of lipophilic ingredients.

The prior art teaches a variety of a water soluble cannabinoid formulations. Examples are shown in Levy, U.S. Pat. No. 10,722,490, and also U.S. Pat. No. 10,568,865 (assigned to Canopy Grown Corporation, of Ontario, Canada), which were granted Jul. 28, 2020 and Feb. 25, 2020, respectively. These patents disclose, a water soluble cannabinoid formulation that includes one or more purified cannabinoids, one or more purified terpenes, and Vitamin E TPGS, in water.

Kuhrts, U.S. Pat. No. 10,328,111, teaches a phytocannabinoid emulsion formulation that includes: a solution of water and propylene glycol; a phytocannabinoid oil; a non-ionic surfactant. The weight ratio of phytocannabinoid content to non-ionic surfactant can be from 1:10,000 to 1:5.

Bromley, U.S. Pat. No. 9,861,611, claims a formulation of water-soluble derivatives of Vitamin E and soft gel compositions, that include a water-soluble Vitamin E derivative mixture (of a particular composition); a non-polar ingredient; a sugar fatty acid ester; and a binder.

Berl, U.S. 2020/0138772, teaches an emulsion that includes CBD, THC, and an emulsifier such as Vitamin E.

Schaneville, U.S. Pat. No. 10,632,164, claims a narrow emulsion that includes (in water), the following: an extract of cannabis or hemp; a thickening agent (selected from a list of such agents); a flavoring agent (selected from a list of such agents); a taste masking agent (selected from a list of such agents); a plasticizer (selected from a list of such agents); a sweetener; a film forming agent; a stabilizing agent; and a binder.

Goskonda, U.S. Pat. Nos. 10,265,293 and 8,222,292, teach oral cannabinoid formulations, including an aqueous-based oral dronabinol solution, that are stable at room or refrigerated temperatures and may possess improved in vivo absorption profiles with faster onset and lower inter-subject variability.

Eades, U.S. 2020/0093787, describes a variety of compositions for administering cannabinoid, including edible forms of cannabinoids. The compositions include Vitamin E, glycerin, and lecithin enriched with phospholipids. Phospholipids and waxes may be used to control the onset, timing of cannabis drug effects.

Schwarz, U.S. 2020/0022386, teaches a method of preparation of beverages, containing5 poorly water soluble cannabinoids, by two-stage dilution of the self-nanoemulsifying concentrate, and composition of the concentrate for preparation of such beverages.

Friedman, U.S. 2019/0298683, teaches self-emulsifying, high concentration and high dose cannabinoid compositions and formulations, to improve administration of cannabinoids and standardized marijuana extracts to patients..

Goskonda, U.S. 2019/0192428, teaches oral cannabinoid formulations, including an aqueous-based oral dronabinol solution, that are stable at room or refrigerated temperatures and may possess improved in vivo absorption profiles with faster onset and lower inter-subject variability.

Levy, U.S. 20190090527, teaches a cannabis composition that includes an acid and base for facilitating the transmucosal absorption of a cannabinoid into the body. In some embodiments, the compositions comprise various combinations of cannabinoids and/or terpenes.

### SUMMARY OF THE INVENTION

The present invention teaches certain benefits in construction and use which give rise to the objectives described below.

The present invention provides a method for forming an emulsion containing a cannabinoid. The method includes the steps of first providing a composition comprising water, vitamin E TPGS, a cannabinoid, and a carrier oil, in a high pressure container. The composition is initially opaque. The composition is then heated to a temperature between 50-120 degrees Celsius, the heating increasing the pressure in the high pressure container to greater than 1 ATM. The high pressure container is then cooled. while mixing, until the composition turns clear, and a micelle water emulsion is formed with particles under 100 nm in particle size.

A primary objective of the present invention is to provide a method for forming an emulsion containing a cannabinoid having advantages not taught by the prior art.

Another objective is to provide a method for forming an emulsion containing a cannabinoid that includes heating a novel composition, under pressure, followed by cooling the composition to form the emulsion.

A further objective is to provide a novel method that provides superior results which are stable for long periods of time.

Other features and advantages of the present invention will become apparent from the following more detailed description of the invention.

### DETAILED DESCRIPTION OF THE INVENTION

The invention includes a cannabinoid emulsion composition, and method of manufacture, that provide superior qualities and

Cannabinoid Emulsion Composition

The cannabinoid emulsion composition includes a cannabinoid and a carrier oil that is selected to have high amounts of long chain triglycerides (LCT), which are emulsified with Vitamin E TPGS (emulsifier non-ionic antioxidant surfactant) in water (e.g., distilled water) using a novel manufacturing process described in greater detail below. The selected carrier oil(s) act as a lipid stabilizer, as discussed in greater detail below. The carrier oil preferably includes at least 10% linoleic acid, in some embodiments having linoleic acid in an amount greater than 50% (by area).

The cannabinoid emulsion composition may further include oil-based forms of Vitamins A, C, D, E, and K, as well as antioxidants and FDA approved food coloring. Examples of these vitamins within the context of this disclosure include: Vitamin A can exist in three forms: retinol, retinal, and retinoic acid. Forms of Vitamin C might include magnesium ascorbyl phosphate, L-ascorbic acid, Tetrahexyldecyl ascorbate, ascorbyl palmitate, ascorbyl glucosamine, and ascorbyl tetraisopalmitate. Vitamin D3. Vitamin K is a family of naphthoquinone compounds comprising K1 (phylloquinone) and several fon is of K2 (MKs, menaquinones).

Vitamin E

There are eight basic forms of the entire fat soluble vitamin E molecule, which are either synthetically or naturally derived. The most typical forms are d-alpha-tocopherol, d-alpha-tocopherol acetate, dl-alpha tocopherol, and dl-alpha tocopherol acetate. The “d” prefix in front of the “alpha” indicates that the product was derived from natural sources, such as vegetable oils or wheat germ; the “dl” prefix indicates that the vitamin was created from a synthetic base. Vitamin E tocopherol, the term tocopherol refers to any naturally occurring or synthetic form of vitamin E, and can refer to a single compound or a mixture. Examples of tocopherols include, for example, a-tocopherol, D-a-tocopherol, β-tocopherol, γ-tocopherol, and δ-tocopherol.

Antioxidants

The emulsions disclosed herein can also contain an antioxidant. The antioxidant can be present, in the continuous phase and/or the dispersed phase. Suitable examples of antioxidants include, but are not limited to, a phenolic compound, a plant extract, or a sulphur-containing compound.

In certain examples disclosed herein the antioxidant can be ascorbic acid or a salt thereof, e.g., sodium ascorbate. In other examples, the antioxidant can be vitamin E, CoQ₁₀, tocopherols, lipid soluble derivatives of more polar antioxidants such as ascobyl fatty acid esters (e.g., ascobyl palmitate), plant extracts (e.g., rosemary, sage and oregano oils, green tea extract), algal extracts, and synthetic antioxidants (e.g., BHT, TBHQ, ethoxyquin, alkyl gallates, hydroquinones, tocotrienols).

Vitamin E TPGS

U.S. Pat. No. 5,798,333, which is hereby incorporated by reference, discloses a method of making a water dilutable, clear cyclosporine-in-water emulsion using tocophersolan (Vitamin E TPGS) as the emulsifier. This patent discloses the use of excess (7.5 times) emulsifier to dissolve cyclosporine. Cyclosporine has a low molecular weight and better solubility than various long chain PUFAs. The finished product is a semi-solid non-flowing gel contained in two-piece gelatine capsule. International publication WO2009/117152 discloses a water dilutable, clear non-polar rianoemulsion containing Vitamin E TPGS, omega-3 triacylglycerol fish oil, and water. The ratio of emulsifier to fish oil is from 1.6:1 (w/w) to 6:1 (w/w). Also, additional components including benzyl alcohol, propylene glycol, glycerol, phospholipids, and a gum based emulsion stabilizers are used. Tocopheryl polyethylene glycol succinate (TPGS). TPGS is a synthetic amphiphile that undergoes enzymatic cleavage to deliver the lipophilic antioxidant, alpha-tocopherol (vitamin E) to cell membranes, and is FDA approved as a water-soluble vitamin E nutritional supplement and drug delivery vehicle.

TPGS, a succinylated polyethylene glycol-bound derivative of vitamin E that retains the same properties of a-tocopherol but is water soluble. Water solubility allows for more reproducible quantitative experimental conditions. Further, TPGS is already used in the pharmaceutical industry as a wetting, emulsification, solubilization, and spreading agent. TPGS can solubilize water-soluble and lipophilic molecules, forming various types of micelles and increasing the solubility of drugs like cyclosporines, taxanes, steroids, and antibiotics.

Suitable examples of Vitamin-E derived emulsifiers include, but are not limited to, polyethylene glycol (PEG) derivatives of tocopherol, such as tocopherol polyethylene glycol diesters (TPGD). A preferred emulsifier is tocopherol polyethylene glycol succinate (TPGS). TPGS analogs, TPGS homologs, and TPGS derivatives are also suitable. Other examples of emulsifiers include tocopherol sebacate polyethylene glycol, tocopherol dodecanodioate polyethylene glycol, tocopherol suberate polyethylene glycol, tocopherol azelate polyethylene glycol, tocopherol citraconate polyethylene glycol, tocopherol methyl citraconate polyethylene glycol, tocopherol itaconate polyethylene glycol, tocopherol maleate polyethylene glycol, tocopherol glutarate polyethylene glycol, tocopherol glutaconate polyethylene glycol, and tocopherol phthalate polyethylene glycol, among others.

(TPGS), such as a TPGS-1000 and/or a d-a TPGS. In another example, the emulsifier is a TPGS analog. TPGS analog refer to compounds, other than TPGS, that are similar to a parent TPGS compound, but differ slightly in composition, for example, by the variation, addition or removal of an atom, one or more units (e.g., methylene unit(s)-(CH2)n) or one or more functional groups. TPGS analogs include Vitamin E derived surfactants, including PEG derivatives of Vitamin E, including vitamin E PEG diesters, such as, but not limited to, tocopherol polyethylene glycol sebacate (PTS), tocopherol polyethylene glycol dodecanodioate (PTD), tocopherol polyethylene glycol suberate (PTSr), tocopherol polyethylene glycol azelate (PTAz), and polyoxyethanyl tocotrienyl sebacate (PTrienS) as well as other PEG derivatives of Vitamin E.

The cannabinoid emulsion composition may further include CoQ₁₀, which is a powerful anti-oxidant. in some embodiments, the CoQ₁₀ may be used in place of the carrier oil. Coenzyme Q₁₀, also known as ubiquinone, is a coenzyme family that is ubiquitous in animals and most bacteria. In humans, the most common form is Coenzyme Q₁₀ or ubiquinone-10.

For purposes of this application, the term “cannabinoid” is defined to include one or more of the known cannabinoid molecules, including but not limited to THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), CBD isolate, full spectrum CBD, broad spectrum CBD, THC distillate, THC isolate, and equivalent materials. Cannabinoids are insoluble in water; but they are soluble in non-polar solvents (e.g. fat and oil). Cannabinoids require a suitable emulsification process to overcome the immiscibility of cannabinoids in water.

For purposes of this application, the te “Vitamin E TPGS” is defined to include any form of Vitamin E (Tocofersolan) that is suitable for emulsifying cannabinoids, including but not limited to any known chemical forms (alpha-, beta-, gamma-, and delta-tocopherol). Commercial examples of suitable forms include ISODEL™ (sold by PMC Isochem, Inc.) TPGS analogues with Polyethylene glycol chain molecular weight of 200 300, 400, 1000, 1500, 2000 and 4000 g/mol., and ASTACANTHIN™ or TOCOPHEROL™, (sold by Antares Health Products, Inc.), and any other commercially available products of this nature. While these examples are provided, the scope of the invention is not limited to these forms, but include any forms known to those skilled in the art.

In addition to these base ingredients, we add, in some embodiments, essential oil(s), terpenes, and/or equivalent molecules as well. Examples of terpenes within the context of this disclosure include, but are not limited to, the following: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Borneol, Bornyl Acetate, Butanoic/Butyric to Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Hemiarin, Hexanaldehyde, Hexanoic Acid, Humulene (Alpha-Humulene) (Beta-Humulene), Ionol (3-oxo-alpha-ionol) (Beta-lonol), Ionone (Alpha-Ionone) (Beta-Ionone), Ipsdienoi, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, ValeraldehydePentanal, Verdoxan, Alpha-Ylangene, Umbelliferone, or Vanillin. Within the context of this disclosure, the term terpene includes the .alpha.-(alpha), .beta.-(beta), .gamma.-(gamma), oxo-, isomers, or any combinations thereof.

In one embodiment, the purified terpene is chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

As used herein, the term “purified” means extracted, isolated, and/or separated from other compounds, formulations, compositions, matter, and/or mass. In one embodiment, the term “purified” refers to a cannabinoid that is separated from the plant matter from which it was derived. In one embodiment, the term “purified” refers to a cannabinoid (a “purified cannabinoid”) that is separated from other cannabinoids that were present in the plant matter from which it was derived. In one embodiment, the term “purified” refers to a cannabinoid (a to “purified cannabinoid”) that is separated from terpenes that were present in the plant matter from which it was derived. In one embodiment, the term “purified” refers to a cannabinoid (a “purified cannabinoid”) that is separated from secondary compounds that were present in the plant matter from which it was derived. In one embodiment, the term “purified” refers to a cannabinoid (a “purified cannabinoid”) that is separated from all material that was present in the plant matter from which it was derived.

In one embodiment, the term “purified” refers to a terpene (a “purified terpene”) that is separated from other cannabinoids that were present in the plant natter from which it was derived. In one embodiment, the term “purified” refers to a terpene (a “purified terpene”) that is separated from terpenes that were present in the plant matter from which it was derived. In one embodiment, the term “purified” refers to a terpene (a “purified terpene”) that, is separated from secondary compounds that were present in the plant matter from which it was derived. In one embodiment, the term “purified” refers to a terpene (a “purified terpene”) that is separated from all material that was present in the plant matter from which it was derived.

For purposes of this application, the term “carrier oil” is defined to include vegetable oils that are pressed from the fatty portions (seeds, nuts, kernels). Examples of carrier oils include, but are not limited to, the following: Acai palm oil, Almond oil, Apricot oil, Argan oil, Arnica oil, Avocado oil, Babassu oil, Barbary Fig Seed oil, Baobab oil, Black Cumin Seed oil, Black Currant seed oil, Black Raspberry seed oil, Blackberry seed oil, Blueberry seed oil, Borage seed oil. Brazil nut oil, Buriti oil, Calendula oil, Camellia seed oil. Canola, Carapa Io oil, Carrot seed oil, Cashew oil, Castor oil, Chardonnay Grape seed oil, Cherry Kernel oil, Chia seed oil, Cloudberry seed oil, Cocoa butter oil, Coconut oil, Corn Oil, Cottonseed oil, Cranberry seed oil, Cucumber seed oil, Elderberry seed oil, Emu oil, Evening primrose oil, Fenugreek oil, Flaxseed/Linseed, Goji Berry seed oil, Grape seed oil, Graviola oil, Guava seed oil, Hazelnut oil, Hemp seed oil, Jambu oil, Jojoba oil. Kukui nut oil, Linseed oil, Macadamia oil, Manketti nut oil, Manila oil, Meadowfoam seed oil, Melon seed oil, Milk Thistle seed oil, Moringa oil, Mustard oil, Neem oil, Olive oil, Palm oil, Passion fruit oil, Peach oil, Peanut oil, Pecan oil, Perilla oil, Pistachio oil, Plum Kernel oil, Pomegranate oil, Poppyseed oil, Pracaxi oil, Prickly Pear seed oil or Barbary Fig, Pumpkin seed oil, Red Raspberry seed oil, Rice bran oil, Rosehip oil, Safflower, Sea Buckthorn oil, Safflower oil, Salicornia oil, Sesame seed oil, Solarium oil, Soybean oil, Strawberry seed oil, Sunflower seed oil, St John's Wort Oil, Sweet Almond oil. Tamanu oil, Tomato seed oil, Trauma oil, Vegetable oil, Vigna mango oil, Walnut oil, Watermelon seed oil, Wheat germ oil, and similar or equivalent oils.

For purposes of this application, the term “essential oil” is defined to include oils that are distilled from the aromatic leaves, bark, and roots of plants, and which have a concentrated aroma, such as is commonly known to those skilled in the art. Examples of suitable essential oils include, but are not limited to, the following: bergamot, blood orange, neroli essential oil, peppermint essential oil, spearmint essential oil, lavender essential oil, lemongrass essential oil. In some embodiments, the cannabis oil extract includes one or more added essential oils including but not limited to the following: Sweet Orange (Citrus sinensis spp), Peppermint (Mentha piperita spp), Lemon (Citrus limon spp), Lavender (Lavendula angustifolia spp) and Vanilla (Vanilla planifolia spp), Agarwood; Agarwood Attar; Ahibero; Allspice; Almond, bitter; Amber Oil; Ambrette Seed; Amyris; Angelica Root; Angelica Seed; Aniseed; Anise; Anise (star); Armoise (Mugwort); Artemisia vestita; Asafoetida; Bakul, Balsam of Peru Oil; Balsam of Peru Resin; Balsamite; Baobab Oil; Basil, Sweet ct Linalool; Basil, Sweet ct Linalool—Organic; Basil, Sweet ct Methyl Chavicol—Organic; Bay; Beeswax; Bergamot; Birch; Boldo; Boronia; Black Cumin; Black Currant Bud; Blue Lotus Attar; Broom; Buchu; Bupleurum (Bupleurum fruticosum); Buddha wood; Butter; Cabreuva; Cade; Cajuput; Calamus: Calendula: Camomile (or Chamomile); Camphor; Cananga; Cangerana; Cape Chamomile (Ericephalus punctulatus) S. Africa, Wild Harvest; Cape May; Caraway; Caraway; Cardamom; Carnation; Carrot Seed; Cascarilla; Cassia; Cassie; Catnip; Cedar (Cedrus) India; Cedarwood; Cedarwood, Atlas—Organic; Cedarwood, Himalayan; Cedarwood, Texas; Cedarwood, Virginia; Celery leaf: Celery Seed; Chamomile, Blue; Chamomile; Chamomile, Roman (Anthemis nobilis); Champa Attar (Michelia champaca) India; Champaca; Chaste tree; Cilantro; Cinnamon; Cinnamon Bark; Cistus; Cistus (Cistus ladaniferus) Corsica; Citronella; Clary Sage Absolute; Clary Sage, Bulgaria; Clary Sage, Russia; Clary Sage, USA; Clementine; Clove; Clove Bud; Cacao; Coconut Pulp; Coffee Bean Oil; Cognac, Green; Coleus; Combava (fruit or leaf); Copaiba; Coriander; Coriander Seed; Cucumber Hydrosol; Cumin; Cumin Seed; Cypress Leaf; Cypress, Blue; Davana; Dill; Elemi; Eucalyptus, Blue Gum; Eucalyptus, Blue Malice; Eucalyptus, Lemon; Fennel (Foeniculum vulgare) Bulgaria; Fennel, Sweet; Fenugreek; Fern (sweet); Fleabane; Fir Needle; Fir, Balsam; Fir, Douglas; Fir, Silver; Fragonia; Frankincense, India; Frankincense, Somalia; Frankincense Frereana; Frankincense, Oman; Frankincense, Oman; Frankincense, Somalia; Galangal; Galbanum; Geranium; Geranium, Egypt; Geranium, Rose; Geranium, South Africa; Ghandi root; Ginger; Ginger Lily; Ginger, Fresh; Gingergrass (Cymbopogon martini); Goldenrod; Grapefruit, Pink; Grapefruit, Ruby Red; Grapefruit, White; Hay; Helichrysum, Albania; Helichrysum, Croatia; Hina Attar, India; Hop; Hyssop Decumbens; Hyssop; Immortelle; Jasmine Absolute, Egypt; Jasmine Absolute, India; Jasmine Concrete; Jasmine; Jasmine Sambac; Jatamansi, (Nardostachs jatamansi) Juniper; Juniper Berry (Juniperus communis) or leaf; Kaffir Lime; Kava Kava; Labdanum; Larch needle; Laurel (Laurus nobilis) Corsica; Laurel Leaf; Lavandin, Grosso; Lavender—High Elevation; Lavender—Wild; Lavender Absolute; Lavender Hydrosol; Lavender, Bulgaria; Lavender, France; Lavender, Maillette; Leleshwa; Lemon; Lemon Tea Tree; Lemon verbena; Lemongrass; Lentisque (Pistacia lentiscus) Corsica; Lime; Lime Essence Oil; Lime, Distilled; Liquidambar (Styrax); Longoza; Lotus Absolute, Pink; Lotus Absolute, White; Lovage leaf Lovage root; Magnolia flower; Mandarin; Mandarin, Green; Mandarin, Red; Mandarin, Yellow; Mango ginger; Marjoram; Manila oil; Melissa; Mint; Mint, Himalayan (Mentha arvensis); Mitti Attar; Motia Attar (Jasmine sambac) India; Mugwort; Mustard; Myrrh; Myrtle, Green; Myrtle (Myrtus Communis); Nagarmotha (Cypriot); Neem (Azadirachta indicd) India; Neroli; Niaouli; Nutmeg; Nut grass; Oakmoss Absolute; Oakwood; Opopanax, Sweet Myrrh (Commiphora guidotti); Orange, Blood; Orange, Sweet; Orange, Wild; Orange Blossom; Orange Essence Oil; Orange, Bitter Green; Orange, Bitter Red; Oregano; Orris Butter; Osmanthus Absolute; Palmarosa; Palmarosa, Nepal; Palmarosa, Sri Lanka; Palo Santo (Bursera graveolens); Palo Santo; Patchouli; Absolute; Patchouli, Dark; Patchouli, Light; Patchouli, Sri Lanka; Pennyroyal; Pepper, Black; Peppercorn, Pink; Peppermint, Chocolate; Peppermint, France; Petitgrain Absolute; Petitgrain Bigarade; Petitgrain sur Fleurs; Petitgrain, Mandarin; Pimento; Pine; Pinion Juniper Co-distillation, Colorado, Wild Harvest; Pinon Pine (Pinus edulis) Colorado, Wild Harvest; Pitta blend (Lavender, Rose Geranium, Ruh Khus); Plai; Pomegranate Seed; Rhododendron (Rhododendron anthopogon); Rhododendron Leaf; Rosalina; Rose; Rose Attar; Rose de Mai Absolute; Rose de Mai Concrete; Rose de Mai Organic Extract; Rose geranium; Rose Hip Seed; Rose Otto, Bulgaria; Rose Otto, Turkey; Rose Otto, White—Organic; Rose vetiver; Rosemary Antioxidant; Rosemary et Cineole; Rosemary ct Verbenone; Rosewood; Rue; Ruh Khus (Vetiveria zizaniodes); Saffron Attar, India; Sage; Samphire (Cristhmum maritimum) Corsica; Sandalwood; Sandalwood, New Caledonia; Sandalwood, Australian—Premium; Sandalwood (Santalum spicatum), Australia; Sandalwood Oil, Royal Hawaiian (Santalum paniculatum); Sandalwood, Royal Hawaiian; Sassafras; Savitri Rose Perfume; Sea Buckthorn; Seaweed; Sierra Juniper (Juniperus occidentalis); Spearmint; Spearmint (Mentha Spicatd) Israel; Spikenard; Spikenard, Green; Spruce, Black; Spruce (Picea mariand) Canada; St. John's Wort (Hypericum perforatum) Bulgaria; Tagetes; Tamanu (Foraha) Oil; Tangelo; Tangerine; Tangerine Murcott; Tansy; Tansy, Blue; Tarragon; Tea Tree; Tea Tree (Leptospermum citratum), Lemon Scented; Tea Tree (Melaleuca altemifolid) South Africa; Thuja; Thyme; Thyme ct Linalool; Tobacco; Tonka Bean; Tuberose; Tulsi, Holy Basic Oil (Ocimum sanctum), Turmeric; Vanilla; Vanilla Bourbon; Verbena; Vetiver—Double Distilled; Vetiver, El Salvador; Vetiver, Haiti; Vetiver, Sri Lanka; Violet Leaf; White Fir (Abies concolor), White Lotus Attar; White Sage (Salvia apiana), Wild Carrot, Corsica; Wintergreen; Wintergreen; Yarrow; Yarrow, Blue Ylang Ylang; and Yuzu.

Essential oils can also provide antioxidant and preservative properties in the cannabis oil compositions. The identity and amount of the essential oil(s) added can depend in part on factors including the strain of cannabis that, has been extracted and the desired organoleptic properties. In general, the amount of total essential oils added to a cannabis extract will range from about 0.01% (w/w) to about 10% (w/w) or more. The % (w/w) values indicated are based on the amount of essential oil added to the amount of total cannabis extract (including vitamin E or additives other than the essential oil, if applicable).

In one embodiment, the purified cannabinoid is contained within a micelle of Vitamin E TPGS. As used herein, the term “micelle” refers to a collection of molecules arranged alongside one another in a generally spherical form often having a pocket inside. In one embodiment, the micelle comprises a lipid molecule. In one embodiment, the lipid molecule comprises both a hydrophobic and hydrophilic region. Micelles may contain as many as 100 molecules or more. In one embodiment the micelle is in a solvent. In one embodiment, the hydrophilic region is in contact with surrounding solvent, sequestering the hydrophobic region in the micelle center. In one embodiment, the micelle is in water and the polar group is on the outside and a hydrophobic end sequesters inside the spherical shape. In one embodiment, the micelle is a reverse micelle, i.e.,, the hydrophilic region of a molecule is surrounded by a nonpolar solvent resulting in a water in oil system. In one embodiment, the reverse micelle comprises hydrophobic groups extended away from the center while hydrophilic groups are sequestered inside the spherical shape. As used herein, the term “contained within” refers to molecules, e.g. cannabinoids and/or terpenes, that are sequestered inside a spherical shape formed by micelles and reverse micelles. In one embodiment, a cannabinoid contained within a micelle allows said cannabinoid to disperse or dissolve within an aqueous formulation.

As used herein, the term “clear” is intended to relation to a solution that is free of visible particles of undissolved compound. A clear solution includes solutions as well as very fine dispersions that remain clear upon sitting undisturbed for one hour or more. Essentially no visible (to the naked eye) particles or micelles are present.

These formulations are then used to create a variety of different products in liquid or solid form, that are ingestible, sublingual tinctures, beverages, soft gels, chewables, tablets, toothpaste, topical skin care products, or administered in any manner known in the art. The products are useful, for example, as a sleep aid, a treatment for anxiety, for pain relief, and topical products for pain/muscle, skin regeneration, skin protection, skin healing, etc. While some examples of final products are described, this should be considered exemplary in nature, and not a limiting list, as those skilled in the art may adapt these processes to a wide variety of final products.

Heat and High Pressure Manufacturing Process

A high pressure container is used in this process so that the process occurs under pressure that is above 1 ATM. In this embodiment, we use a glass bottle with a screw top lid. This mixture is heated by a heat source, in this case a 1,000 watt microwave oven, to a temperature between 50-120 C, (optimal temperature is between 80-100 C). This heating in the high pressure container has the effect of increasing the pressure of the mixture to above 1 ATM.

The bottle is removed from microwave oven, at which point the ingredients inside the bottle are opaque in color. The bottle is swirled or shaken lightly to make sure all the ingredients are well mixed and have an even temperature throughout the entire mix. The bottle may or may not be vented when removed from the microwave oven. The bottle is then swirled under cool, running water, or placed in an ice bath and swirled until the opaque emulsion turns crystal clear, which means you have a micelle water emulsion with particles under 100 nm in particle size; this is the goal we are trying to achieve. Dynamic Light Scattering (DLS) testing has shown particle size ranging from 20-80 nanometers, perfect size for intracellular uptake or cell endocytosis.

A wide variety of different water emulsions have been prepared, using a combination of different Cannabinoids and or Terpenes with a combination of Carrier Oils or just one type of Carrier Oil, Distilled Water, and Vitamin E TPGS. These emulsions may further include essential oils, and Coenzyme CoQ₁₀. Coenzyme CoQ₁₀ has been found to also replace Vitamin E in some emulsions. These emulsions, stored in their original high pressure bottles for 1½ years in storage, are still stable, and none of the oils have separated. After doing a lot of research and experimenting, we believe that the Long Chain Triglycerides (LCT) and/or fatty acids found in Carrier Oils, act as stabilizers. Thus, we prefer to use Carrier Oils that are high in LCT and Linoleic Acid. Amounts of different, individual Carrier Oils and combination of different Carrier Oils, work a little faster in the conversion process from opaque to a clear.

Water emulsions using heat and pressure, which include a cannabinoid, water, Vitamin E TPGS, and carrier oil, can be heated in a high-pressure bottle in a hot water bath, on a cook top, or in a microwave oven. The various ingredients are combined in the high-pressure vessel (e.g., glass bottle) until it reaches a suitable temperature (80-100 C). The bottle is then removed from the heat source (e.g., microwave), the contents are swirled or lightly shake until all the ingredients are homogenous and the temperature is even throughout the entire emulsion.

The high-pressure glass bottle is then placed in an ice bath or under cool running water, and the bottle is mixed (e.g., swirled or stirred) until the cloudy/opaque emulsion turns crystal clear; continue swirling until temperature drops to 35-55 C.

Various formulations are discussed in the following examples; however, these are illustrative of the invention, and should not be considered limiting, but the invention includes equivalent formulations consistent with the teachings of the current disclosure.

Formula 1) Distilled water 91.0 grams, CBD Isolate 4.7 grams, Vitamin E TPGS 22 grams, Carrier Oils (Grapeseed and Olive oil) 8.2 grams. There is a minimum amount of Carrier Oil that must be used or it will not turn into a clear nanoemulsion. If you use 0 to 6 grams of Carrier Oil, this formula will not turn into a clear nanoemulsion; it will stay cloudy/opaque in color. When the amount of CBD isolate is increased, the amount of Carrier Oil must be increased. Example: Distilled Water 92 grams, CBD Isolate 5.3 grams, Vitamin TPGS 22 grams, Carrier Oil 10.8 grams. The amount of Carrier Oil grams, Vitamin E TPGS grams, and Distilled Water grains is dependent upon the starting amount of Cannabinoid being used.

Formula 2) Distilled water 87.0 grams, THC Distillate 4.6 grams, Vitamin E TPGS 21 grams, Carrier Oil (Olive Oil) 5.5 grams. In this example, as in the first formula, there is a minimum amount of Carrier Oil that must be used or it will not turn into a clear nanoemulsion. If you use 0 to 4 grains of Carrier Oil, this formula will not turn into a clear nanoemulsion; it will stay cloudy/opaque in color. The amount of Carrier Oil grains, Vitamin E TPGS grams, and Distilled Water grams is dependent upon the starting amount of Cannabinoid being used.

Formula 3) Distilled water 90.0 grams, THC Distillate 5.89 grams, Vitamin E TPGS 23 grams, and Carrier Oil (Hemp Oil) 7.0 grams.

Formula 4) Distilled Water 66.1 grams. THC Distillate 4.2 grams, Vitamin E TPGS 21 grains, Carrier Oil (California Olive Oil) 4.2 grams. This is a special Olive Oil that is very high in Linoleic Acid.

Formula 5) Distilled Water 87.0 grams, THC Distillate 4.6 grams, Vitamin E TPGS 24.0 grams, Green Crack Terpene Mixture 0.23 grams, and Carrier Oil (California Olive) 5.7 grams.

Formula 6) Distilled water 86 grams, CoQ₁₀ 8.6 grams, TPGS 26.0 grams (note, NO Carrier Oil was used). So far, this is the only oil-based ingredient that doesn't need a Carrier Oil. A Carrier Oil (Hemp Oil) in the amount of 1 gram may also be used, and the emulsion changed into a clear nanoemulsion. It is therefore possible to omit the use of a carrier oil if CoQ₁₀ is included in its stead.

Formula 7) Distilled water 91.0 grams, CBD Isolate 4.7 grams, Vitamin E TPGS 22 grams, Carrier Oil (California Olive) 8.2 grams, and Organic Peppermint Oil 4.7 grams.

Formulas 1, 2, 3, 4, 5, 7 will not work unless there is a carrier oil(s). If you use only distilled water, CBD Isolate and Vitamin E TPGS, the emulsion will stay cloudy/opaque after being cooled; the emulsion will not turn clear. If you use only distilled water, THC Distillate and TPGS, the emulsion will stay cloudy/opaque after cooling; the emulsion will not turn clear. Having clarity, means you can see through the emulsion with clarity, the particle size is sub 100 nanometers.

The starting amount of the cannabinoid(s), determines the amount/ratio of Vitamin E TPGS, carrier oil(s) and distilled water needed to create a clear nanoemulsion. There is a ratio formula for each ingredient. These ratios will have a range a little higher and a little lower than the example below.

Formula 1 for example: Distilled Water 91.0 grams, CBD Isolate 4.7 grams, TPGS 22 grams, Carrier Oil(s) (Grapeseed 4.1 grams and Olive 4.1 grams).

CBD Isolate 4.7 g×7.7446808=8.2 g of Carrier Oil(s)

CBD Isolate 4.7 g+8.2 g Carrier Oil=12.9 g ×1.7054263=22 Vitamin E TPGS

CBD Isolate 4.7 g+8.2 g Carrier Oil+22 Vitamin TPGS=34.9×2.607449=91 g H2O

It has also been found, however, in other embodiments, that the water amount may be reduced by up to 40% and still form a suitable emulsion.

You can mix water emulsions together when cooled to create one emulsion with higher concentrations of different active oils. You can take these oil-based water emulsions and add them directly to any other liquid, and it will disperse and become a homogeneous mixture; it is very stable. After the emulsion is made, you can also add water-based active ingredients, such as vitamin C, Hyaluronic acid, etc., into the water emulsion. You can spray directly onto your skin or hair and it will be absorbed extremely fast. You can place under your tongue for faster absorption. You can take two different oil-based, water emulsions and mix them together to create one emulsion with higher concentrations of different active oils and then add them into another liquid or spray them onto your skin, hair, or place under your tongue.

As used in this application, the words “a,” “an,” and “one”are defined to include one or more of the referenced item unless specifically stated otherwise. The terms “approximately” and “about” are defined to mean +/−10%, unless otherwise stated. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by claims made to the invention.

What is claimed is:

1. A method for forming an emulsion containing a cannabinoid, the method comprising the steps of:
providing a composition comprising water, vitamin E TPGS, a cannabinoid, and a carrier oil, in a high pressure container, the composition, being opaque;
heating the composition to a temperature between 50-120 degrees Celsius, the heating increasing the pressure in the high pressure container to greater than 1 ATM; and
cooling the high pressure container, while mixing, until the composition turns clear, and a micelle water emulsion is formed with particles under 100 nm in particle size.

2. The method of claim 1, wherein the carrier oil is high in long chain triglycerides (LCT) and includes at least 10% linoleic acid.

3. The method of claim 1, wherein the carrier oil is high in long chain triglycerides (LCT).

4. The method of claim 1, wherein the carrier oil includes at least 10% linoleic acid.

5. The method of claim 1, wherein the carrier oil includes grapeseed oil, olive oil, or hemp oil.

6. The method of claim 1. wherein the cannabinoid includes CBD or

7. The method of claim 1, wherein the composition is heated to a temperature of between 80-100 degrees Celsius.

8. The method of claim 1, wherein the step of cooling the high pressure container, while mixing, is continued until the temperature of the composition drops to 35-55 C.

9. The method of claim 1, wherein the composition further includes at least one terpene.

10. The method of claim 9, wherein the at least one terpene includes one of the following: 7,8-dihydro-alpha-ionone, 7,8-dihydro-beta-ionone, Acetanisole, Acetic Acid, Acetyl Cairene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Bomeol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovornifoliol, Diallyl Disulfide, Dihydroactinidiolide, Dimethyl Disulfide, Eicosane/Icosane. Elemene (Beta-Elemene), Estragole, Ethyl acetate, Ethyl Cinnamate, Ethyl to maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaieine), Gurjunene (Alpha-Gurjunene), Hemiarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid. Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Neryl, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol,Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol. Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

11. The method of claim 1, wherein the cannabinoid includes at least one of the following: THC (tetrahydrocannabinol), THCA (tetrahydrocannabinolic acid), CBD (cannabidiol), CBDA (cannabidiolic acid), CBL (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivatin), CBCV (cannabichromevarin), CBGV (cannabigerovarin), CBGM (cannabigerol monomethyl ether), CBE (cannabielsoin), CBT (cannabicitran), CBD isolate, full spectrum CBD, broad spectrum CBD, THC distillate, THC isolate

12. The method of claim 1, wherein the carrier oil includes at least one of the following: Acai palm oil, Almond oil, Apricot oil, Argan oil. Arnica oil, Avocado oil, Babassu oil, Barbary Fig Seed oil, Baobab oil, Black Cumin Seed oil, Black Currant seed oil, Black Raspberry seed oil, Blackberry seed oil, Blueberry seed oil, Borage seed oil, Brazil nut oil, Buriti oil, Calendula oil, Camellia seed oil, Canola, Carapa oil, Carrot seed oil, Cashew oil, Castor oil, Chardonnay Grape seed oil, Cherry Kernel oil, Chia seed oil, Cloudberry seed oil, Cocoa butter oil, Coconut oil, Corn Oil, Cottonseed oil, Cranberry seed oil, Cucumber seed oil, Elderberry seed oil, Ernu oil, Evening primrose oil, Fenugreek oil, Flaxseed/Linseed, Goji Berry seed oil, Grape seed oil, Graviola oil, Guava seed oil, Hazelnut oil, Hemp seed oil, Jambu oil, Jojoba oil, Kukui nut oil, Linseed oil, Macadamia oil, Manketti nut oil, Manila oil, Meadowfoam seed oil, Melon seed oil, Milk Thistle seed oil, Moringa oil, Mustard oil, Neem oil, Olive oil, Palm oil, Passion fruit oil, Peach oil, Peanut oil, Pecan oil, Perilla oil, Pistachio oil, Plum Kernel oil, Pomegranate oil, Poppyseed oil, Pracaxi oil, Prickly Pear seed oil or Barbary Fig, Pumpkin seed oil, Red Raspberry seed oil. Rice bran oil, Rosehip oil, Safflower, Sea Buckthorn oil, Safflower oil, Salicornia oil, Sesame seed oil, Solarium oil, Soybean oil, Strawberry seed oil, Sunflower seed oil, St John's Wort Oil, Sweet Almond oil, Tamanu, oil, Tomato seed oil, Trauma oil, Vegetable oil, Vigna mungo oil, Walnut oil, Watermelon seed oil, and Wheat germ oil.

13. A method for forming an emulsion containing a cannabinoid, the method comprising the steps of:
providing a composition comprising water, vitamin E TPGS, a cannabinoid, and a carrier oil that includes at least 10% linoleic acid, in a high pressure container, the composition being opaque;
heating the composition to a temperature between 80-100 degrees Celsius, the heating increasing the pressure in the high pressure container to greater than 1 ATM; and
cooling the high pressure container, while mixing, until the temperature of the composition drops to below 55 C.

14. The method of claim 13, wherein the composition further includes at, least, one terpene.

15. The method of claim 14, wherein the at least one terpene includes one of the following: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptl/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Gerrnacrenes, Germacrene B, Guaia-1(10),11-diene Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isobomeol, Isomyrcenol, Isopulegol, Isovaleric Acid, Isoprene, Kahweol Lavandulol, Limonene, Gamma-Linolenic Acid, Linalook Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol. Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol. Terpine-4-ol, Alpha-Terpinene, Gamma-Terpinene, Terpinolene, Thiophenol, Thujone, Thymol, Alpha-Tocopherol, Tonka Undecanone, lindecanal, Valeraldehyde/Pentanal, Verdoxan, Alpha-Ylangene, Umbelliferone, or Vanillin.

16. The method of claim 13, wherein the cannabinoid includes at least one of the following: THC (tetrahydrocannabinol), THCA (tetrahydrocannabinolic acid), CBD (cannabidiol), BDA (carmabidiolic acid), CCN (cannabinol), CBG (cannabigerol), CBC (cannabichromene), CBL (cannabicyclol), CBV (cannabivarin), THCC (tetrahydmcannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), CBD isolate, full spectrum CBD, broad spectrum CBD, THC distillate, THC isolate

17. The method of claim 13, wherein the carrier oil includes at least one of the following: Acai palm oil, Almond oil, Apricot oil, Argan oil, Arnica oil, Avocado oil, Babassu oil, Barbary Fig Seed oil, Baobab oil, Black Cumin Seed oil, Black Currant seed oil, Black Raspberry seed oil, Blackberry seed oil, Blueberry seed oil, Borage seed oil, Brazil nut oil, Buriti oil, Calendula oil, Camellia seed oil, Canola, Carapa oil, Carrot seed oil, Cashew oil, Castor oil, Chardonnay Grape seed oil, Cherry Kernel oil, Chia seed oil, Cloudberry seed oil, Cocoa butter oil, Coconut oil, Corn Oil, Cottonseed oil, Cranberry seed oil, Cucumber seed oil, Elderberry seed oil, Emu oil, Evening primrose oil, Fenugreek oil, Flaxseed/Linseed, Goji Berry seed oil, Grape seed oil, Graviola oil, Guava seed oil, Hazelnut oil, Hemp seed oil, Jambu oil, Jojoba oil, Kukui nut oil, Linseed oil, Macadamia oil, Manketti nut oil, Marula oil, Meadowfoam seed oil, Melon seed oil, Milk Thistle seed oil, Moringa oil, Mustard oil, Neem oil, Olive oil, Palm oil, Passion fruit oil, Peach oil, Peanut oil, Pecan oil, Perilla oil, Pistachio oil, Plum Kernel oil, Pomegranate oil, Poppyseed oil, Pracaxi oil, Prickly Pear seed oil or Barbary Fig, Pumpkin seed oil, Red Raspberry seed oil, Rice bran oil, Rosehip oil, Safflower, Sea Buckthorn oil, Safflower oil, Salicomia oil, Sesame seed oil, Solarium oil, Soybean oil, Strawberry seed oil, Sunflower seed oil, St John's Wort Oil, Sweet Almond oil, Tamanu oil, Tomato seed oil, Trauma oil, Vegetable oil, Vigna mungo oil, Walnut oil, Watermelon seed oil, and Wheat germ oil.
